(19) United States
(12) Patent Application Publication
(10) Pub. No.: US 2026/0096962 A1
(43) Pub. Date: Apr. 9, 2026

(54) ACTIVE PREMIX FOR ANTIPERSPIRANT PRODUCTS

(71) Applicant: THE PROCTER & GAMBLE COMPANY, Cincinnati, OH (US)

(72) Inventors: Wade Charles HENKE, Mason, OH (US); Shawn David MCCONAUGHY, Cincinnati, OH (US); David Frederick SWAILE, Cincinnati, OH (US)

(21) Appl. No.: 19/349,072

(22) Filed: Oct. 3, 2025

Related U.S. Application Data
(60) Provisional application No. 63/702,962, filed on Oct. 3, 2024.
(60) Provisional application No. 63/875,781, filed on Sep. 4, 2025.

(51) Int. Cl. A61K 8/26; A61K 8/04; A61K 8/20; A61K 8/44; A61Q 15/00

(52) U.S. Cl. CPC A61K 8/26; A61K 8/046; A61K 8/20; A61K 8/44; A61Q 15/00

(57) ABSTRACT

A particulate active premix composition that includes basic aluminum chloride of formula Al₂(OH)_{3.778}Cl_{2.222} to Al₂(OH)_{5.048}Cl_{0.952}, an amino acid, an alkaline earth metal salt, and greater than 1% of an alkali metal salt and/or magnesium chloride. The composition can be a particle. The particulate active premix can be incorporated into antiperspirant products.

### FIELD OF THE INVENTION

The present disclosure relates to an antiperspirant composition and methods relating thereto. More particularly, the antiperspirant composition is a plurality of particles that includes a basic aluminum chloride salt of formula Al₂(OH)_{3.778}Cl_{2.222} to Al₂(OH)_{5.048}Cl_{0.952}, an amino acid, greater than 1% of an alkali metal salt and/or magnesium chloride, and optionally an alkaline earth metal salt.

### BACKGROUND OF THE INVENTION

Antiperspirants are widely used personal care products designed to reduce perspiration and body odor. These formulations primarily work by delivering active ingredients that temporarily block the sweat glands, thereby reducing the amount of sweat that reaches the skin's surface. The most common active ingredients in antiperspirants are aluminum-based compounds, such as aluminum chloride and aluminum zirconium (Al—Zr) salts. These compounds react with the electrolytes and proteins in sweat to form a gel-like plug that inhibits sweat excretion.

Despite their widespread use of Al—Zr -based actives, there are reasons to limit the use of zirconium in formulations. Zirconium compounds can be expensive and may face regulatory restrictions, including safety concerns about inhaling aerosolized zirconium compounds, which led to bans on their use in aerosol spray products.

As alternatives, aluminum-based antiperspirant actives, such as the basic aluminum chlorides: aluminum chlorohydrate (ACH), aluminum sesquichlorohydrate (ASCH), and aluminum dichlorohydrate (ADCH) are available. However, these alternatives tend to have lower efficacy as compared to aluminum-zirconium (Al—Zr) compounds.

The hygroscopicity of aluminum-only active ingredients can be enhanced through various treatments, such as thermal activation and/or incorporation of amino acids and alkaline earth metal salts. However, if aluminum-based salts are too hygroscopic, they can easily absorb moisture from the air, turning the powder sticky and intractable, and in more extreme cases, into an unmanageable gel or liquid, making it challenging to use these salts in antiperspirant products.

Therefore, there is a need for an aluminum-based antiperspirant active with optimized hygroscopicity, as well as methods relating thereto.

### SUMMARY OF THE INVENTION

A particulate active premix composition comprising: (a) a basic aluminum chloride salt of formula Al₂(OH)_{3.778}Cl_{2.222} to Al₂(OH)_{5.048}Cl_{0.952}; (b) an amino acid; (c) an alkaline earth metal salt; (d) greater than 1% of an alkali metal salt and/or magnesium chloride; and wherein the composition comprises a plurality of particles.

A method of making a particulate antiperspirant active premix comprising: (a) providing a basic aluminum chloride salt with the formula Al₂(OH)_{3.778}Cl_{2.222} to Al₂(OH)_{5.048}Cl_{0.952}; (b) adding calcium chloride to provide a calcium to aluminum atomic ratio of at least 0.05 and adding an amino acid to provide a glycine to calcium molar ratio from about 0.5 to 5 to form an aqueous solution; (c) adding an alkali metal solution to the aqueous solution at a concentration of more than 0.5% to form the aqueous premix solution; (d) heating the aqueous premix solution at a temperature of more than 60° C. for more than 1 hour; (e) after step (d), drying the aqueous premix solution to a plurality of particles, forming the particulate antiperspirant active premix; (f) optionally grinding the particulate antiperspirant active premix to a Dv50 particle size less than 50 microns; and (g) optionally adding water to regenerate a solution comprising the aqueous premix solution.

A method of making a particulate antiperspirant active premix comprising: (a) providing an aqueous solution of aluminum chlorohydrate Al₂(OH)_{3.778}Cl_{2.222} to Al₂(OH)_{5.048}Cl_{0.952}; (b) adding calcium chloride to provide a calcium to aluminum atomic ratio of at least 0.05 and adding glycine to provide a glycine to calcium molar ratio from about 1 to 2 to form an aqueous solution; (c) heating the aqueous solution at a temperature of more than 60° C. and less than 150° C., for more than 1 hour; (d) after step (c), adding sodium chloride at a concentration of more than 0.5% of the aqueous solution; (e) after step (d), drying, preferably spray drying, the aqueous solution to form a plurality of particles to form the particulate antiperspirant active premix; (g) optionally grinding the particulate antiperspirant active premix to a Dv50 particle size less than 50 microns; and (h) optionally adding water to regenerate a solution comprising the aqueous premix solution.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 shows turbidity as a function of dilution for active premix solutions with different amino acids;

FIG. 2A shows an image from an SEM microscope of Aluminum Chlorohydrate (ACH);

FIG. 2B shows an image from an SEM microscope of Active Premix Example 1B;

FIG. 2C shows the change in mass vs. time for Active Premix Examples 1A (0% NaCl), 1B (5% NaCl), and 1C (10% NaCl);

FIG. 3A shows an image from an SEM microscope of Active Premix Example 2A;

FIG. 3B shows an image from an SEM microscope of Active Premix Example 2B;

FIG. 3C shows the change in mass vs. time for Active Premix Examples 2A (0% NaCl), 2B (5% NaCl), and 2C (10% NaCl);

FIG. 4A shows an image from an SEM microscope of Active Premix Example 3A;

FIG. 4B shows an image from an SEM microscope of Active Premix Example 3B;

FIG. 4C shows the change in mass vs. time for Active Premix Examples 3A (0% NaCl), 2B (5% NaCl), and 2C (10% NaCl);

FIG. 5 an image from an SEM microscope of Active Premix Example 4;

FIG. 6 an image from an SEM microscope of Active Premix Example 5;

FIG. 7 an image from an SEM microscope of Active Premix Example 6; and

FIG. 8 is a representative chromatogram for Peaks I-V.

### DETAILED DESCRIPTION OF THE INVENTION

Aluminum-based salts are widely used as active ingredients in AP products. Many products use aluminum-zirconium (Al—Zr) salts. However, aluminum-only active ingredients may be preferred due to factors such as cost, regulatory constraints, and safety concerns related to zirconium inhalation in aerosol formulations. While aluminum-only actives exist, they typically exhibit lower efficacy and lower pH compared to their Al—Zr counterparts.

The effectiveness of aluminum-only actives can be improved through various treatments. One such method involves heating the active solution prior to spray drying, which enhances the concentration of smaller, more effective polymers within the formulation. In both aluminum-only and Al—Zr complexes, these smaller polymers are commonly referred to as band 3 or peak 4 (U.S. Pat. Nos. 4,359,456 and 10,526,210 describe heating aluminum active solutions). The relative concentration of these polymers is often measured by the band III/II ratio or the Peak 4/3 ratio, with values exceeding 0.75 generally indicating a more potent active ingredient. This process, known as “activation,” results in formulations that are characterized as “activated,” “enhanced,” or “improved” antiperspirant actives.

Various companies have attempted to develop effective aluminum-only antiperspirant actives. Procter & Gamble® introduced an activated aluminum chlorohydrate solution in Old Spice® Ever Clear, a water-based stick with a calcium to aluminum atomic ratio of 0.06 and a glycine to calcium molar ratio of 5.2 for stability. However, consumer preference for the dry feel of anhydrous products limited its acceptance. Additionally, attempts to create a particulate active premix via spray drying aluminum chlorohydrate, calcium chloride, and glycine were unsuccessful, as the mixture lacked sufficient hygroscopic properties for effective dissolution in sweat. Unilever®'s Degree® Advanced Antiperspirant Dry Spray includes aluminum sesquichlorohydrate activated with calcium chloride and glycine, which offers superior hygroscopicity for rapid dissolution. However, the increased acidity of these actives poses risks of corrosive damage to metal components within aerosol dispensers and can degrade fragrance components.

It was found that a particulate active premix containing basic aluminum chloride salts of formula Al₂(OH)_{3.778}Cl_{2.222} to Al₂(OH)_{5.048}Cl_{0.952} (Al:Cl atomic ratios between about 0.90 and about 2.10) and amino acids, an alkali metal salt, and optionally an alkaline earth metal salt can have enhanced hygroscopicity and antiperspirant efficacy. Additionally, the active premix can have higher pH values compared to other hygroscopic actives, which can improve compatibility with a wide array of perfumes, additives, anhydrous formulas, processing equipment, and containers.

Basic aluminum chlorides have the general formula Al_{y}(OH)_{3y-z}Cl_{z}·nH₂O. Of particular interest are the basic aluminum chloride salts of formula Al₂(OH)_{3.778}Cl_{2.222} to Al₂(OH)_{5.048}Cl_{0.952}. This includes aluminum chlorohydrate (ACH), aluminum sesquichlorohydrate (ASCH), and/or aluminum dichlorohydrate (ADCH), which are detailed in Table 1 below. Other characteristics of ACH, ASCH, and ADCH can be found in the United States Pharmacopoeia and National Formulary (USP/NF) in its monograph (2024).

TABLE 1
Basic Aluminum Chlorides

| | | Aluminum to | | |
|---|---|---|---|---|
| Compound | | Chloride Atomic | Chemical Formula | |
| Name | Abbreviation | Ratio (Al:Cl) | Range | Example |
| Aluminum | ACH | 1.91-2.10 | Al₂(OH)_{5.048}Cl_{0.952} to | Al₂(OH)₅Cl |
| Chlorohydrate | | | Al₂(OH)_{4.953}Cl_{1.047} | |
| Aluminum | ASCH | 1.26-1.90 | Al₂(OH)_{4.947}Cl_{1.053} to | Al₂(OH)_{4.5}Cl_{1.5} |
| Sesquichlorohydrate | | | Al₂(OH)_{4.413}Cl_{1.587} | |
| Aluminum | ADCH | 0.90-1.25 | Al₂(OH)_{4.400}Cl_{1.600} to | Al₂(OH)₄Cl₂ |
| Dichlorohydrate | | | Al₂(OH)_{3.778}Cl_{2.222} | |

Each particle in the active premix can include aluminum and the alkali metal salt and/or magnesium chloride. The alkali metal or magnesium can be interdispersed with the aluminum and/or it can be in a surface coating that can be visualized using energy-dispersive X-ray (EDX) spectroscopy coupled with a scanning electron microscope (SEM). For instance, the alkali metal salt can be sodium chloride and each particle can contain aluminum, sodium, and chloride. The sodium can be interdispersed with the aluminum and chloride and/or the sodium can be present in a surface coating that can be visualized using an SEM. Similarly, each particle can contain aluminum, magnesium, and chloride. The magnesium can be interdispersed with the aluminum and chloride or it can be present in a surface coating that can be visualized using an SEM

The active premixes can have an amino acid to alkaline earth metal molar ratio of <6, ≤5.5, ≤5.25, ≤5, ≤4.9, <3, ≤2, ≤1.75, ≤1.5, or ≤1.4. The active premixes can have an amino acid to alkaline earth metal molar ratio of ≥0.25, ≥0.5, ≥0.75, ≥0.90, ≥1.0, ≥1.1, ≥1.25, or ≥1.3. Without wishing to be bound by theory, it is believed that lower ratios can be more effective in producing active premixes with improved hygroscopic properties. On the other hand, higher ratios are believed to be beneficial in achieving longer periods of solution stability before drying the active premix to a particle and it can be desirable to adjust the ratio based on manufacturing conditions.

The active premix can have a pH of about 3.5 to about 5.2, from about 3.6 to about 5.0, from about 3.7 to about 4.9, or from about 3.8 to about 4.8, as determined by the pH Test Method, described herein. The active premix can have a pH ≥3.5, ≥3.6, ≥3.75, or ≥4.0.

The active premix can have a maximum change in mass over 48 hours of ≥45%, ≥47%, ≥50%, ≥55%, ≥60%, ≥52%, ≥54%, ≥60%, ≥70%, or ≥80% according to the Water Vapor Sorption Method, described herein. In order to maintain the stability and processability required for practical application in manufacturing antiperspirant formulations and shipping of antiperspirant formulations and active salts it can be advantageous for the active premix to have a change in mass over 48 hours of ≤150%, ≤140%, ≤130%, ≤125%, ≤115%, ≤110%, ≤106%, ≤100%, ≤90%, ≤80%, ≤70%, ≤65%, or ≤60% according to the Water Vapor Sorption Test Method, described herein.

The polymer size distribution of the ACH in the active premix can be defined by size exclusion chromatography method using Gel Permeation Chromatography (GPC), described herein in the GPC Test Method. The active premix can have a peak IV/III (Band III/II) ratio of exceeding nb 0.75, ≥1, ≥1.5, ≥2, ≥2.1, ≥2.4, ≥2.5, ≥3, ≥3.25, ≥3.4, ≥3.5, ≥3.75, ≥3.85, ≥3.90, ≥4.0, ≥4.2, ≥4.4, ≥4.6, ≥4.8, ≥5.0, ≥5.2, ≥5.4, ≥5.6, ≥5.8, ≥6.0, ≥6.25, ≥6.5, ≥6.75, ≥7.0, ≥7.25, ≥7.5, ≥7.75, or ≥7.9 according to the GPC Test Method. Active premixes having a peak IV/III ratio exceeding 0.75 can be referred to as “activated” and the process of heating to increase the peak IV/III ratio can be referred to as “activation.” The peak IV/III ratio can be dependent on several factors including the level and type of alkaline earth metal salt, amount and type of amino acid, and temperature and time of heating in solution prior to drying.

Alkaline earth metal salts can include an alkaline earth metal cation and an anion that is acceptable for application to the skin. Alkaline earth metals are elements in Group 2 of the periodic table and include beryllium, magnesium, calcium, and strontium. The anions associated with the alkaline earth metals salts can include chloride, sulfate, nitrate, or other halogen anions. More specifically, the salts can include calcium chloride, calcium nitrate, calcium sulfate, strontium chloride, strontium sulfate and strontium nitrate. Among these, calcium chloride may be preferred. The composition can include from about 0.5% to about 13%, from about 1% to about 10%, from about 1.5% to about 7%, or from about 1.75% to about 5% alkaline earth metal salts.

The amino acids can include natural amino acids, unnatural amino acids (e.g., non-canonical), and/or derivatives thereof (e.g., betaines such as trimethylglycine). The amino acids can include alanine, arginine, asparagine, aspartic acid, beta-alanine, cysteine, glutamic acid, glutamine, glycine, trimethylglycine, histidine, isoleucine, leucine, lysine, methionine, phenylalanine, proline, serine, threonine, tryptophan, tyrosine, valine, or mixtures thereof. Among these, glycine, trimethylglycine, alanine, leucine, or mixtures thereof may be preferred. Among these, glycine, alanine, and/or trimethylglycine may be preferred. The composition can include from about 1% to about 25%, from about 2% to about 20%, or from about 3% to about 18%, of the amino acid.

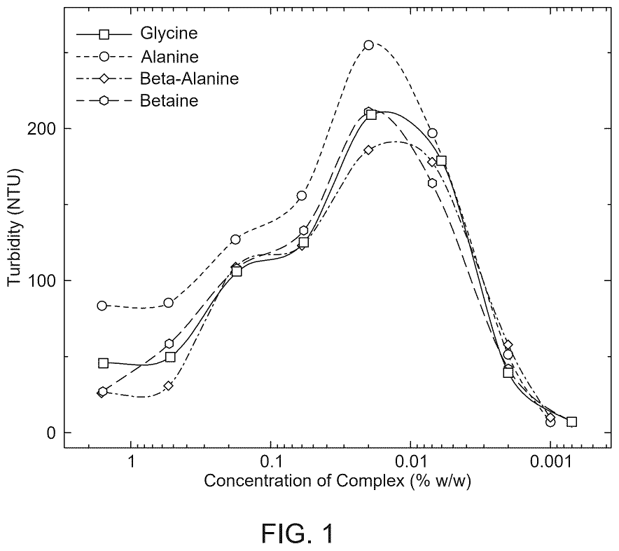

FIG. 1 shows the results of an experiment designed to evaluate the impact of various amino acids on an aluminum chlorohydrate active premix, which included calcium chloride, sodium chloride, and one of four amino acids: glycine, alanine, beta-alanine, or trimethylglycine.

In the experiment, 0.5 g of the active premix (APM) was combined with 10 g of synthetic sweat to create a stock solution. From this stock solution, eight sequential one-third dilutions were performed to prepare the test solutions. Specifically, Dilution 1 consisted of 5 g of the stock solution mixed with 10 g of synthetic sweat. Dilution 2 was made by combining 5 g of Dilution 1 with 10 g of synthetic sweat, and this process was repeated for a total of eight dilutions. Table 2 shows the dilutions.

After completing the dilutions, the test solutions were capped and allowed to equilibrate at ambient conditions for 16 hours. The turbidity of each test solution was then measured using a HACH 2100Q Portable Turbidimeter.

Table and FIG. 1 show the results from this test and indicate that the turbidity serves as a proxy for precipitation in the sweat duct; greater turbidity indicates a more effective test solution. The results revealed that the active premixes containing glycine, alanine, beta-alanine, or trimethylglycine demonstrated similar performance characteristics when tested in vitro. It is believed that these amino acids, along with others, would be acceptable to consumers in the active premixes described herein. Additionally, some consumers may prefer naturally derived amino acids, including trimethylglycine.

TABLE 2

| Alanine | | Beta-Alanine | | Trimethylglycine | | Glycine | |
|---|---|---|---|---|---|---|---|
| APM | | APM | | APM | | APM | |
| Conc. | Turbidity | Conc. | Turbidity | Conc. | Turbidity | Conc. | Turbidity |
| (% w/w) | (NTU) | (% w/w) | (NTU) | (% w/w) | (NTU) | (% w/w) | (NTU) |
| 1.614 | 84 | 1.622 | 26 | 1.588 | 27 | 1.576 | 46 |
| 0.54 | 85 | 0.539 | 31 | 0.53 | 59 | 0.524 | 50 |
| 0.179 | 127 | 0.179 | 109 | 0.175 | 108 | 0.174 | 106 |
| 0.06 | 156 | 0.06 | 123 | 0.058 | 133 | 0.058 | 125 |
| 0.02 | 255 | 0.02 | 186 | 0.02 | 211 | 0.019 | 209 |
| 0.007 | 197 | 0.007 | 178 | 0.007 | 164 | 0.006 | 179 |
| 0.002 | 51 | 0.002 | 58 | 0.002 | 42 | 0.002 | 39 |
| 0.001 | 7 | 0.001 | 10 | 7.00E−04 | 7 | 7.00E−04 | 7 |

Moreover, the active premixes can have an alkaline earth metal to aluminum atomic ratio ≥0.03, ≥0.05, ≥0.06, ≥0.09, or ≥0.10. The active premixes can have an alkaline earth metal to aluminum atomic ratio of ≤1, ≤0.75, ≤0.5, ≤0.4, ≤0.35, ≤0.25, or ≤0.2. The active premixes can have an alkaline earth metal to aluminum atomic ratio of about 0.03 to about 0.5, about 0.06 to about 0.35, or about 0.7 to about 0.25. Without wishing to be bound by theory, it is believed that higher ratios can create more activation or allow activation at lower temperatures. This higher ratio may also provide longer solution stability of the polymers before the active premix is dried to a particle. However, the higher ratio can generate higher absolute levels of glycine in the active premix that may, in turn, lead to a less hygroscopic active premix.

The active premixes can also include an alkali metal salt that is acceptable for use on skin. Alkali metals are elements in Group 1 of the periodic table and can include: lithium, sodium, potassium, or mixtures thereof. Anions for these salts of these metals can include chloride, sulfate, phosphates, nitrate, other halogen anions, or mixtures thereof. Acceptable salts can include sodium chloride, sodium sulfate, sodium phosphate, sodium nitrate, lithium chloride, lithium sulfate, lithium phosphate, lithium nitrate, potassium chloride, potassium nitrate, potassium sulfate, potassium phosphate, or mixtures thereof. The salts can include sodium chloride, potassium chloride, lithium chloride, or mixtures thereof. Among these, sodium chloride may be preferred. Without wishing to be bound by theory, it is believed that salts can further increase the water dissolution rate of the active premix. These salts can comprise up to 25% of the active premix, less than 15%, or less than 10%. If present, these salts can comprise at least 1% of the active premix, at least 2%, at least 3%, at least 4%, or at least 5%.

The active premixes can be made according to the following method: the basic aluminum chloride is present or dissolved in an aqueous solution that also contains the amino acid, and the alkaline Earth metal, if present. Optionally, the solution can be heated to be activated, which is indicated by the having a peak IV/III ratio of exceeding 0.75. The time and temperature of heating will be dependent on the concentration of basic aluminum chloride in solutions during heating, the alkaline earth metal mole ratio, and the amino acid to alkaline earth metal molar ratio. Heating temperatures are 70° C. to 95° C. and heating time range from 1-24 hours. Heating temperatures, heating times, the alkaline earth metal atomic ratio, and the amino acid to alkaline earth metal molar ratio can be varied based on desired peak IV/III ratio, desired degree of hygroscopicity and available processing equipment. If present, the alkali metal salt can be added before, after, or independent of heating.

The particulate active premixes can be dried by any suitable manner including belt drying, tray drying and spray drying. Among these, spray drying may be preferred. The dried particle may be ground to desired particle size and/or sieved to desired particle distribution and may be spherical, platelet or random mixture in shape. The particle size may vary based on the final product (e.g., spray, cream, roll-on or stick product). The particulate active premix could be generally spherical in shape and can have a Dv50 average particle size of 25-35 microns. The particulate active premix could be generally platelet in shape and have a Dv50 average particle size of 12 to 18 microns. The active could be finely ground to have more than 85% or more than 90% (Dv90) of the particle having a size less than 10 microns and an average of less than 6 microns. Optionally, the active premix powder can also be dissolved or suspended in a solution, including aqueous, propylene glycol, propylene carbonate, or alcohol-based solution.

### Methods of Use

The active premix particles can be incorporated into antiperspirant products. The product can be a spray, a roll-on, cream, paste, foam, wipes, or a stick including anhydrous sticks, gel sticks including aqueous gel sticks, soft solids, or glycol-based sticks. The antiperspirant product may be topically applied to the axilla or other area of the skin in any known or otherwise effective method for controlling malodor associated with perspiration. These methods comprise applying to the axilla or other area of the human skin an effective amount of the antiperspirant product that includes the active premix composition, typically about 3.5 to about 5 mg/cm² of the product, and more typically about 4 mg/cm². The product is generally a leave-on composition that can provide lasting odor protection and freshness and the composition may provide 72 hours or more of odor control after application. The product can provide wetness protection, which can refer to the product's ability to help reduce or prevent excessive sweating and the resulting wetness under the arms.

### Definitions

Except as otherwise noted, the articles “a”, “an”, and “the” mean “one or more”.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

As used herein, “visual detection” means that a human viewer can visually discern if the had deliquesced with the unaided eye (except for standard corrective lenses adapted to compensate for near-sightedness, farsightedness, or astigmatism, or other corrected vision) in lighting at least equal to the illumination of a standard 100-watt incandescent white light bulb at 30 cm.

All numbers expressing pH values are to be understood as being modified by the term “about,” and as encompassing readings using a pH meter having a variation of up to +10%, such as up to +9%, up to +8%, up to +7%, up to +6%, up to +5%, up to +4%, up to +3%, up to +2%, or up to +1%, which a skilled person will recognize relates to the inherent variation in pH meters.

As used herein, the expressions “ranging from” and “between” are inclusive of the endpoints of the recited range(s).

All percentages are by weight of the composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified.

When a range of values is described herein, the range is contemplated to encompass the boundaries of the range as well as each value and sub-range within that range and individual values within that range. For example, “2 mg/L to 6 mg/L” is contemplated to encompass, for example, 2.0 mg/L, 2.5 mg/L, 2.7 mg/L, 3 mg/L, 4 mg/L, 4.5 mg/L, 5 mg/L, 5.5 mg/L, 6 mg/L, 2 mg/L to 3.5 mg/L, 2.5 mg/L to 4 mg/L, 2.5 mg/L to 4.5 mg/L, 4.5 mg/L to 6 mg/L, 4.8 mg/L to 5.3 mg/L, and so forth.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### Test Methods

### pH Test Method

The pH of particulate active premixes and/or the actives are determined by creating a 15% solution of the powder in deionized water and measuring with a calibrated glass electrode and meter such as a Mettler Toledo® SevenDirect pH SD20 or equivalent. The pH of the aqueous phase is then determined.

### GPC Test Method

The GPC Test Method is performed on sample active premix to quantify relative concentration of aluminum polymer populations present. The method is preferably performed on active or active premix but can also be performed on finished antiperspirant product if active or active premix is not available. Peaks corresponding to five characteristic polymer populations are identified and integrated, and ratios of relative areas of peaks of interest are calculated and reported.

### Sample Preparation

If sample active or active premix is available, it is dissolved or dispersed in 0.01-M nitric acid such that active is present at 1.0% w/w. If only sample finished antiperspirant product is available, it is dissolved or dispersed in 0.01-M nitric acid such that the active is present at 1.0% w/w. After dilution, the nitric acid solution may be filtered (for example, using a 0.45-μm nylon syringe filter) if necessary. In any case, only the resulting nitric-acid extraction solution is subsequently injected into the chromatograph.

### Apparatus and Procedure

GPC chromatographic separation is performed using 0.01-M nitric acid mobile phase, using a total 900 mm length of silica columns. Exemplary suitable apparatus consists of three consecutive μPorasil Columns, 3.9×300 mm, 10 mm packing (available from Waters, Milford, Massachusetts), plumbed in series. The chromatograph is equipped with a refractive-index detector. Exemplary suitable refractive-index detector is ERC RefractoMax 520 (available from IDEX, Oak Harbor, Washington). A 5-μ L injection volume is used, and an exemplary suitable flow rate is 0.8 mL/min.

### Analysis

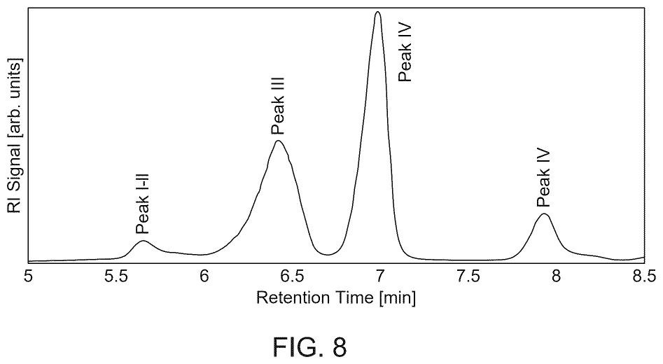

Chromatograms are processed and peaks integrated, for example using suitable software such as Thermo Fisher Scientific Chromeleon Data System. The focus of this analysis is a set of four or five peaks appearing early in the chromatogram that correspond to five characteristic polymer populations. (Sharper peaks, generally with longer retention time, are understood to correspond to small molecules present in the extracted active and are disregarded in this analysis.) The peaks observed in the chromatogram are designated in order of appearance on the chromatogram as Peaks I-II (generally appears as a single peak) and Peaks III, IV and V. A representative chromatogram containing the abovementioned peaks is shown in FIG. 8. The area of Peaks I-II, III, IV and V correspond to the relative concentration of aluminum polymer populations present in the analyzed active in the injected sample. (It is noted that these five polymer populations are generally recognized as present. Swaile U.S. Pat. No. 6,149,897 refers to the peaks in the chromatograms as Peaks I-II, Peak III, Peak IV, and Peak V. Similarly, Gosling U.S. Pat. No. 4,359,456 refers to Peak I-II as “Band 1,” Peak III as “Band 2, Peak IV as “Band 3”, and Peak V as “Band 4.”

Peaks I-II, III, IV, and V are each integrated, and their peak areas (arbitrary units) are recorded. The ratios between combinations of peaks of interest may be calculated and expressed as a dimensionless ratio. For example, the ratio of the area of Peak IV to the area of Peak III is calculated and reported as the “Peak IV/III ratio” to the nearest 0.01.

Water Vapor Sorption Test Method The Water Vapor Sorption Test Method is used to quantify the amount of water vapor uptake by a sample or composition when contained in a warm, moist environment. This method makes use of a dynamic vapor sorption (DVS) instrument to first dry and condition a specimen of a sample active premix or composition and then measure the increase of mass over time as the sample is held at elevated temperature and relative humidity.

A DVS instrument suitable for this method is capable of controlling percent relative humidity (% RH) to within ±5% RH, temperature to within ±2° C., and measuring mass to a precision of ±0.01 mg. It is further capable of maintaining environments of 0% RH at both 37° C. and 50° C. and 90% RH at 37° C. One suitable exemplary apparatus is the S Endeavour Dynamic Vapor Sorption Analyzer (Surface Measurement Systems Ltd.), or equivalent. The DVS instrument is configured to record the mass of specimen at ten-minute intervals with a precision of 0.01 mg or better.

A 20.0±5.0 mg specimen of material is spread evenly on a tared aluminum sample pan appropriate for the DVS instrument. The specimen and pan are introduced into the DVS, and the specimen is first dried at 37° C. and 0% RH for 10 hours. The specimen is then conditioned at 37° C. and 0% RH for 20 hours. The specimen mass point recorded at 37° C. and 0% RH is taken as the initial specimen mass, denoted mᵢ. The specimen is then exposed to an environmental state at 37° C. and 90% RH for 18 hours. For any point in time t during the final 18 hours of the measurement, the percent change in mass of the specimen is calculated by the expression:

$$Percent change in mass [ \% ] = \frac{{m}_{t} - {m}_{i}}{{m}_{i}} \times 100 \%$$

where mₜ is the measured specimen mass at that point in time, and time t is the total time passed after the beginning of the first, drying condition of the DVS. The maximum percent change in mass of the specimen observed over the final 18 hours of the measurement, corresponding to time 30 hours to 48 hours of the DVS measurement procedure overall, is reported as the Change in Mass of the specimen, reported in percent to the nearest 1%.

### Examples

The examples in Table 3 and Table 4 were made as follows:

### ACH Active Premix (APM) Examples:

Aluminum chlorohydrate solution (40% anhydrous active), calcium chloride solution (32% w/w), and glycine were added to a glass reaction vessel equipped with a stir bar. The solution was allowed to stir until homogeneous. Following visible homogeneity, the reaction vessel was placed in a gravity oven and the solution was brought to 95° C. for at least one and up to 24 hours. The reaction vessel was then cooled to room temperature and the solution was isolated. The isolated solution was then divided into parts, and the appropriate amount of sodium chloride was then added to the premix solution to target 0%, 5%, and 10% in the powder product. Before spray drying, the solutions were filtered through a 0.7 m filter. Following this, the resulting solutions were spray dried to isolate the corresponding powder. Spray drying was conducted using either a Buchi Mini Spray Dryer S-290 or a Buchi Mini Spray Dryer S-300 (Model: Corrosives 250° C. Pro) using the following conditions: Drying gas 30.5 m³/h, Spray gas 1200 L/h, Inlet temperature: 220° C., Outlet temperature 115-125° C., once spray drying was completed, the APM was collected in a vessel for further analysis using various analytical methods.

### ASCH Active Premix (APM) Examples

Aluminum sesquichlorohydrate powder (82% anhydrous active), calcium chloride, and glycine were added to a 5 L reactor equipped with a mechanical stirrer, reflux condenser, and temperature controller. The reactor was brought to 95° C. for at least one and up to 24 hours. The reaction vessel was then cooled to room temperature, and the solution was isolated. The isolated solution was then divided into parts, and the appropriate amount of sodium chloride was then added to the premix solution to target 0%, 5%, and 10% in the powder product. Before spray drying, the solutions were filtered through a 0.7 m filter. Following this, the resulting solution was spray dried to isolate the corresponding powder. Spray drying was conducted using either a Buchi Mini Spray Dryer S-290 or a Buchi Mini Spray Dryer S-300 (Model: Corrosives 250° C. Pro) using the following conditions: Drying gas 30.5 m³/h, Spray gas 1200 L/h, Inlet temperature: 220° C., Outlet temperature 115-125° C., once spray drying was completed, the APM was collected in a vessel for further analysis using various analytical methods.

### ADCH Active Premix (APM) Examples

Ca/Gly activated ADCH solution was purchased commercially. The isolated solution was then divided into parts, and the appropriate amount of sodium chloride was then added to the premix solution to target 0%, 5%, and 10% in the powder product. Before spray drying, the solutions were filtered through a 0.7 m filter. Following this, the resulting solution was spray dried to isolate the corresponding powder. Spray drying was conducted using either a Buchi Mini Spray Dryer S-290 or a Buchi Mini Spray Dryer S-300 (Model: Corrosives 250° C. Pro) using the following conditions: Drying gas 30.5 m³/h, Spray gas 1200 L/h, Inlet temperature: 220° C., Outlet temperature 115-125° C., once spray drying was completed, the APM was collected in a vessel for further analysis using various analytical methods.

The pH for the examples in Table 3 and Table 4 were determined according to the pH Test Method, described herein.

The Kinetic Study for the examples in Table 3 and Table 4 was performed according to the Water Vapor Sorption Test Method, described herein. The test determined the amount of water vapor sorption that occurs in a raw material.

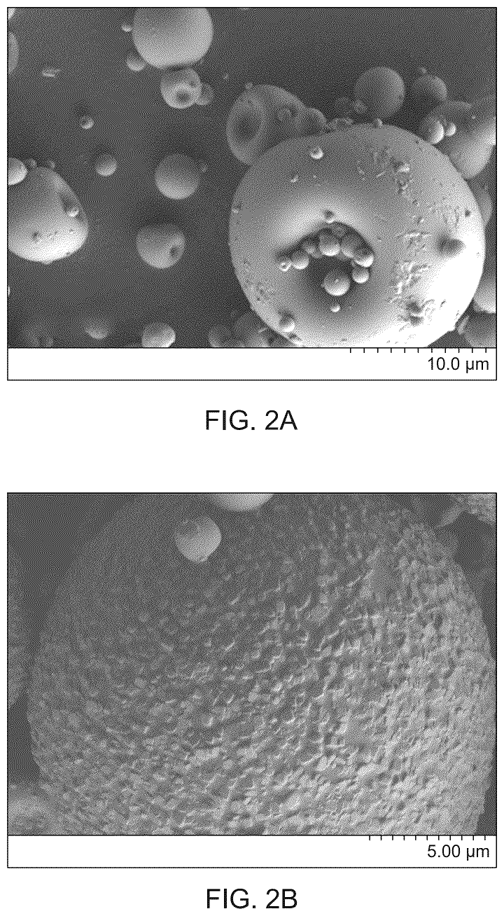

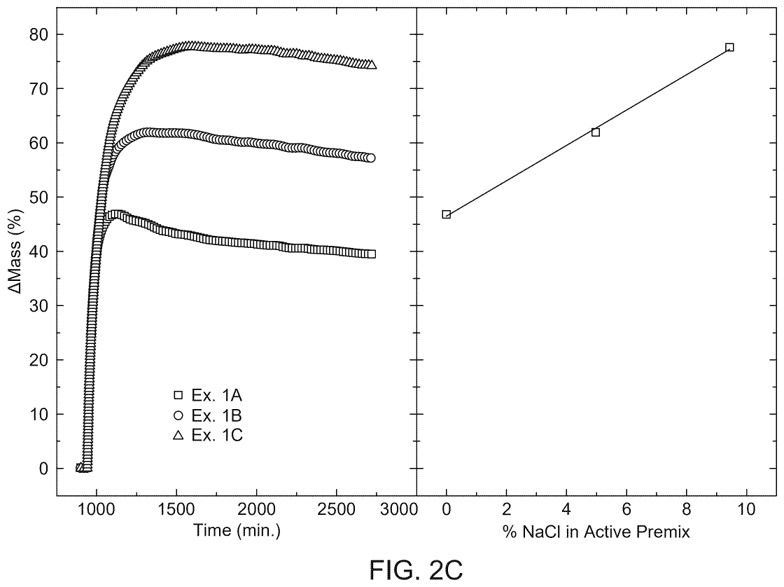

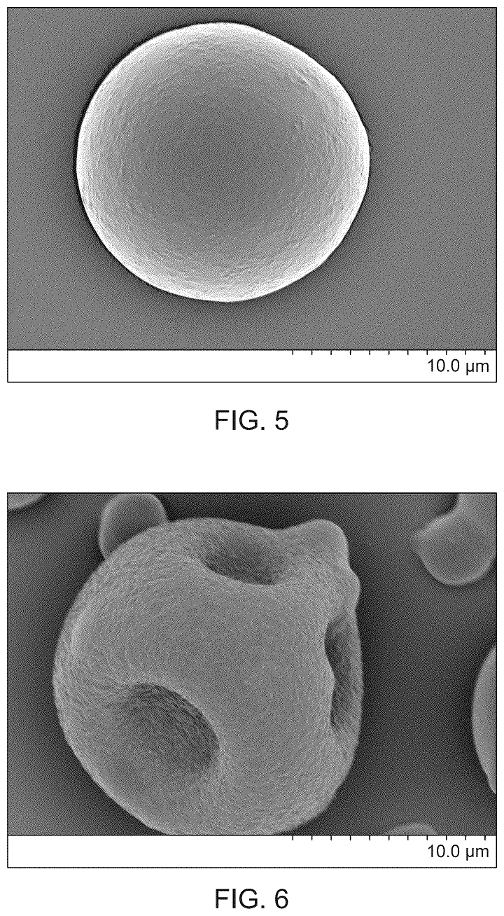

The SEM images shown in FIGS. 2A-2B, 3A-3B, 4A-4B, and 5-7 clearly show the effects of adding sodium chloride to the system. When no sodium chloride is added, the particles appear relatively smooth, as seen in FIGS. 2A, 3A, and 4A. However, after the addition of sodium chloride, the particles exhibit a rougher surface, with visible cubic sodium chloride crystals present, as shown in FIGS. 2B, 3B, 4B, and 5-7. Before spray coating, the particles in FIGS. 5-7 appeared similar to those depicted in FIG. 2A.

It was surprising to find sodium chloride deposits on the particle surfaces, especially since the spray-dried mixture is homogeneous and contains relatively low sodium compared to the amounts of aluminum (with a Na to Al atomic ratio of 0.12-0.28). In some instances, the sodium chloride covers a significant portion of the particle surface (e.g., more than a majority, >80% up to 95%) while in other cases, only about 50% may be covered, as shown in the SEM images.

FIGS. 2C, 3C, and 4C show that as the amount of sodium chloride added increases, the hygroscopicity also increases. These figures also surprisingly show that there is a linear dose response based on the amount of sodium chloride that is added. This indicates that sodium chloride enhances the moisture-absorbing properties of the active premix compared to formulations without sodium chloride, as demonstrated in APM Examples 1A, 2A, and 3A. The increased ability to absorb moisture from the environment due to the added sodium chloride is expected to facilitate a faster dissolution of the active premix in sweat, ultimately enhancing the effectiveness of the antiperspirant.

Additionally, a direct comparison of Examples 1B vs. 1C, 2B vs. 2C, and 3B vs. 3C illustrates that higher sodium chloride content correlates with greater hygroscopicity. However, it is important to recognize that while increased hygroscopicity can enhance the performance of the active premix, there is a trade-off. If the active premix becomes too hygroscopic, it can create challenges in shipping and incorporating it into antiperspirant formulations.

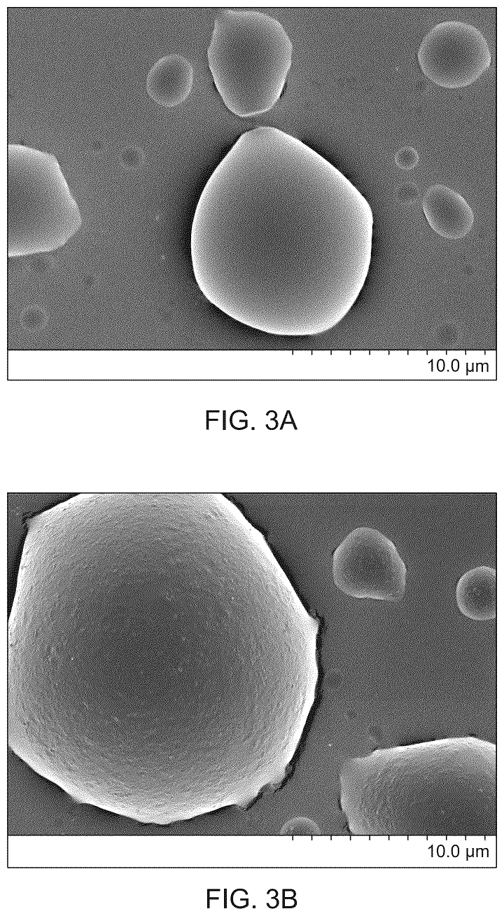

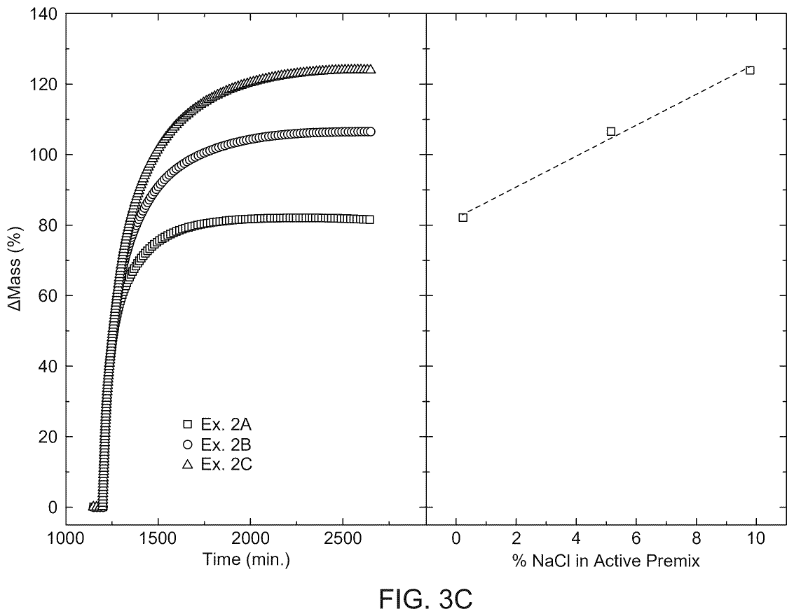

TABLE 3

| | Ex. 1A | Ex. 1B | Ex. 1C | Ex. 2A | Ex. 2B | Ex. 2C |
|---|---|---|---|---|---|---|
| | APM | APM | APM | APM | APM | APM |
| Basic Aluminum | ACH | ACH | ACH | ASCH | ASCH | ASCH |
| Chloride Salt | | | | | | |
| % Al | 21.3 | 19.2 | 18.1 | 17.8 | 17.2 | 16.2 |
| % Cl (Total) | 18.36 | 19.02 | 21.38 | 21.99 | 23.88 | 25.50 |
| % Ca | 2.17 | 1.99 | 1.87 | 3.9 | 3.82 | 3.6 |
| % Glycine | 12.3 | 17.7 | 17.2 | 17.90 | 15.59 | 14.15 |
| Target % Glycine | 20.52 | 20.05 | 19.53 | 19.00 | 18.60 | 18.19 |
| Al:Cl Atomic Ratio | 1.52 | 1.33 | 1.11 | 1.06 | 0.95 | 0.83 |
| Al:Cl Atomic Ratio | 1.93 | 2.02 | 1.93 | 1.56 | 1.62 | 1.61 |
| of Active | | | | | | |
| % Na | 0 | 1.96 | 3.71 | 0.09 | 2.03 | 3.85 |
| Target % NaCl | 0 | 5 | 10 | 0 | 5 | 10 |
| Calculated % NaCl | 0.00 | 4.98 | 9.43 | 0.23 | 5.16 | 9.79 |
| Ca:Al Atomic Ratio | 0.069 | 0.070 | 0.070 | 0.147 | 0.150 | 0.150 |
| Gly:Ca Mol Ratio | 3.03 | 4.75 | 4.91 | 2.45 | 2.18 | 2.10 |
| Active pH | | | | 4.19 | 4.33 | 4.39 |
| Peak 4:3 Ratio | | | | 6.49 | 6.63 | 6.69 |
| SEM Image | | FIG. 2B | | FIG. 3A | FIG. 3B | |

| | | |
|---|---|---|
| Kinetic Study | FIG. 2C | FIG. 3C |

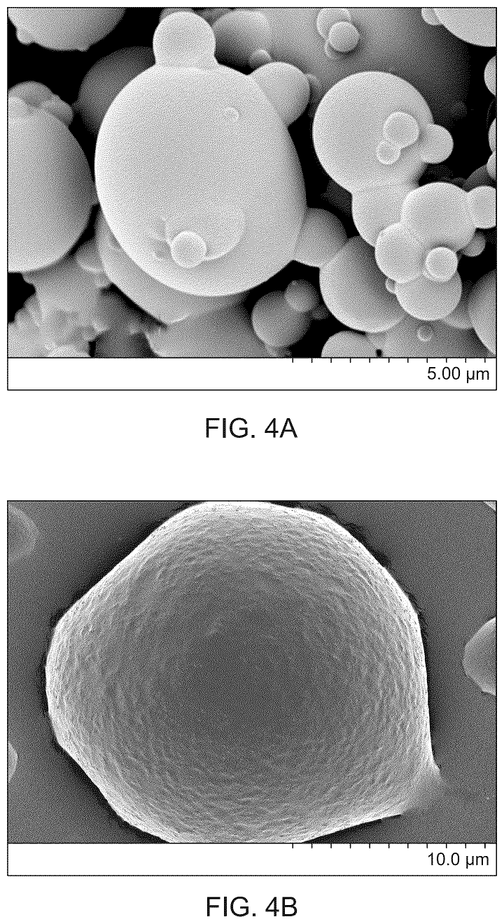

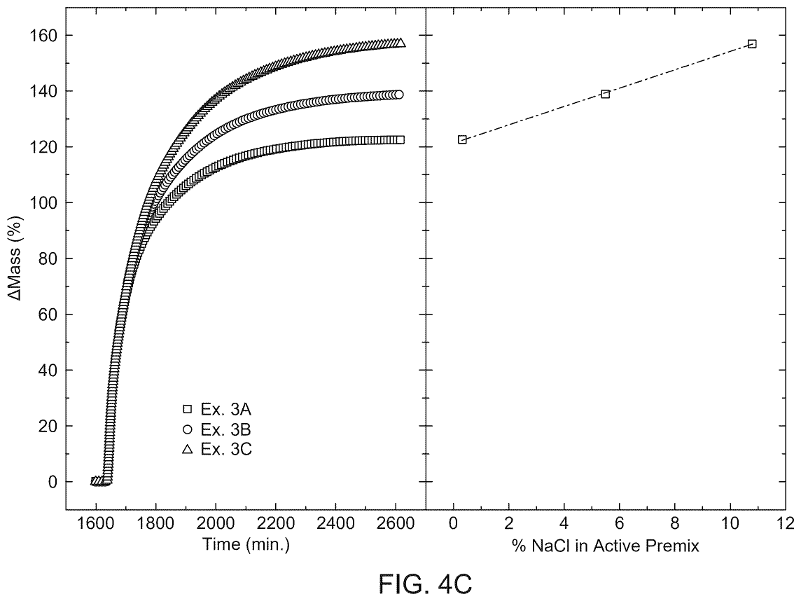

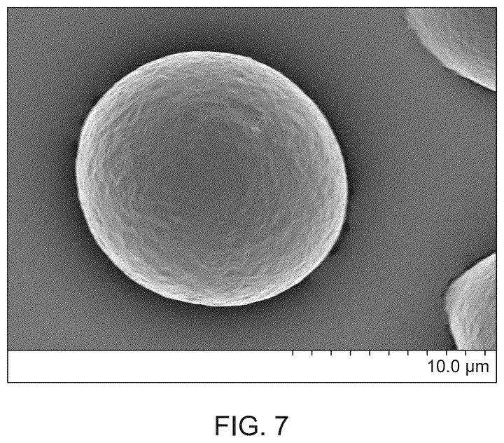

TABLE 4

| | Ex. 3A | Ex. 3B | Ex. 3C | Ex. 4 | Ex. 5 | Ex. 6 |
|---|---|---|---|---|---|---|
| | APM | APM | APM | APM | APM | APM |
| Basic Aluminum | ADCH | ADCH | ADCH | ASCH | ASCH | ASCH |
| Chloride Salt | | | | | | |
| % Al | 19.7 | 18.7 | 17.7 | 18.2 | 18.4 | 17.8 |
| % Cl (Total) | 26.18 | 28.11 | 30.05 | 23.29 | 23.14 | 22.43 |
| % Ca | 2.94 | 2.79 | 2.6 | 4.11 | 4.13 | 3.99 |
| % Glycine | 3.38 | 2.68 | 2.63 | | | |
| % Alanine | | | | 12.21 | | |
| % Beta-Alanine | | | | | 10.31 | |
| % Trimethylglycine | | | | | | 11.45 |
| Target % Amino Acid | 3.60 | 3.52 | 3.43 | 11.82 | 11.82 | 15.59 |
| Al:Cl Atomic Ratio | 0.99 | 0.87 | 0.77 | 1.03 | 1.04 | 1.04 |
| Al:Cl Atomic Ratio | 1.25 | 1.24 | 1.23 | | | |
| of Active | | | | | | |
| % Na | 0.128 | 2.16 | 4.24 | 1.92 | 1.93 | 1.86 |
| Target % NaCl | 0 | 5 | 10 | 5 | 5 | 5 |
| Calculated % NaCl | 0.33 | 5.49 | 10.78 | 4.88 | 4.91 | 4.73 |
| Ca:Al Atomic Ratio | 0.100 | 0.100 | 0.099 | 0.152 | 0.151 | 0.151 |
| Amino Acid:Ca Mol | 0.61 | 0.51 | 0.54 | 1.59 | 1.33 | 1.53 |
| Ratio | | | | | | |
| Active pH | 3.93 | 4.03 | 4.00 | 4.68 | 4.62 | 4.66 |
| Peak 4:3 Ratio | 0.519 | 0.536 | 0.552 | 3.455 | 2.021 | 2.36 |
| SEM Image | FIG. 4A | FIG. 4B | | FIG. 5 | FIG. 6 | FIG. 7 |

| | |
|---|---|
| Kinetic Study | FIG. 4C |

Table 5 presents the maximum moisture absorption over a 48-hour period for the tested samples depicted in FIGS. 2C, 3C, and 4C, following the Water Vapor Sorption Test Method outlined in this document. The results indicate that the active premix samples containing a 10% sodium chloride concentration (e.g., Examples 1C, 2C, and 3C) exhibited superior moisture absorption compared to those with a 5% sodium chloride concentration (e.g., Examples 1B, 2B, and 3B) or no sodium chloride (e.g., Examples 1A, 2A, and 3A).

However, visual detection revealed that Examples 2C (the ASCH example with 10% NaCl) and Examples 3A-C (all ADCH examples) were excessively hygroscopic and had deliquesced, posing challenges for formulation in antiperspirant products. Furthermore, highly hygroscopic actives can lead to issues in aerosol antiperspirant formulations by potentially clogging the exit orifice. Notably, Example 2B (the ASCH sample with 5% NaCl) also showed signs of deliquescence, suggesting that a moisture pickup of approximately 106% may serve the approximate cutoff for ASCH.

There is variability in the hygroscopicity of basic aluminum chloride salts prior to the addition of sodium chloride, indicating that it may be beneficial to explore the addition of alternative salts, such as magnesium chloride, to enhance the usability of ASCH and ADCH in formulations.

TABLE 5

| | Target Added Sodium | Maximum Moisture | Did the APM |
|---|---|---|---|
| | Chloride | Pickup over 48 Hours | example |
| | Concentration (%) | (%) | deliquesce? |
| Ex. 1A ACH APM | 0 | 47 | No |
| Ex. 1B ACH APM | 5 | 62 | No |
| Ex. 1C ACH APM | 10 | 78 | No |
| Ex. 2A ASCH APM | 0 | 82 | No |
| Ex. 2B ASCH APM | 5 | 106 | The example |
| | | | showed signs of |
| | | | deliquescence but |
| | | | is still considered |
| | | | processable and |
| | | | the AP product |
| | | | will likely be |
| | | | consumer |
| | | | acceptable. |
| Ex. 2C ASCH APM | 10 | 124 | Yes |
| Ex. 3A ADCH APM | 0 | 123 | Yes |
| Ex. 3B ADCH APM | 5 | 139 | Yes |
| Ex. 3C ADCH APM | 10 | 157 | Yes |

### Combinations:

A. A particulate active premix composition comprising:
   a. a basic aluminum chloride salt of formula Al₂(OH)_{3.778}Cl_{2.222} to Al₂(OH)_{5.048}Cl_{0.952};
   b. an amino acid;
   c. greater than 1% of an alkali metal salt and/or magnesium chloride; and wherein the composition comprises a plurality of particles.
B. The composition according to Paragraph A, wherein the composition comprises up to 25% of the alkali metal salt, preferably up to 20% alkali metal salt, more preferably up to 15% alkali metal salt, and most preferably up to 10% alkali metal salt.
C. The composition according to Paragraphs A-B, wherein the composition comprises an alkali metal salt chosen from sodium chloride, potassium chloride, lithium chloride, or mixtures thereof.
D. The composition according to Paragraphs A-C, wherein the alkali metal salt comprises sodium chloride.
E. The composition according to Paragraphs A-D, wherein the particulate active premix comprises magnesium chloride.
F. The composition according to Paragraphs A-E, wherein the basic aluminum chloride salt comprises aluminum chlorohydrate salt of formula Al₂(OH)_{5.05}Cl_{0.95} to Al₂(OH)_{4.95}Cl_{1.05}.
G. The composition according to Paragraphs A-E, wherein the basic aluminum chloride salt comprises aluminum sesquichlorohydrate salt having an aluminum to chloride atomic ratio of from 1.26 to 1.90.
H. The composition according to Paragraphs A-E, wherein the basic aluminum chloride salt comprises aluminum dichlorohydrate salt having an aluminum to chloride atomic ratio of from 0.90 to 1.25.
I. The composition according to Paragraphs A-H, wherein the composition comprises from about 1% to about 25%, preferably from about 2% to about 20%, or more preferably from about 3% to about 18% of the amino acid.
J. The composition according to Paragraphs A-I, wherein the amino acid is chosen from alanine, arginine, asparagine, aspartic acid, beta-alanine, trimethylglycine, cysteine, glutamic acid, glutamine, glycine, histidine, isoleucine, leucine, lysine, methionine, phenylalanine, proline, serine, threonine, tryptophan, tyrosine, valine, or mixtures thereof.
K. The composition according to Paragraphs A-J, wherein the amino acid is chosen from glycine, alanine, trimethylglycine, or mixtures thereof.
L. The composition according to Paragraphs A-K, wherein the amino acid is chosen from glycine, alanine, beta-alanine, trimethylglycine, or mixtures thereof.
M. The composition according to Paragraphs A-L, wherein the amino acid is glycine.
N. The composition according to Paragraphs A-M, wherein the composition further comprises an alkaline earth metal salt.
O. The composition according to Paragraph N, wherein the composition comprises from about 0.5% to about 10%, preferably from about 1% to about 7%, more preferably from about 1.5% to about 5%, and even more preferably from about 1.75% to about 4.5% of the alkaline earth metal salt.
P. The composition according to Paragraphs M-O, wherein the alkaline earth metal salt is chosen from calcium chloride, calcium nitrate, calcium sulfate, strontium chloride, strontium sulfate, strontium nitrate, or mixtures thereof.
Q. The composition according to Paragraphs M-P, wherein the alkaline earth metal salt is chosen from calcium chloride, strontium chloride, or mixtures thereof.
R. The composition according to Paragraphs M-Q, wherein the alkaline earth metal salt comprises calcium chloride.
S. The composition according to Paragraphs M-R, wherein the amino acid to alkaline earth metal molar ratio is ≤7, preferably ≤6, and more preferably ≤5.
T. The composition according to Paragraphs M-S, wherein the amino acid to alkaline earth metal molar ratio is >0, preferably ≥0.25, more preferably ≥0.5, even more preferably ≥1.0, and even more preferably ≥1.5.
U. The compositions according to Paragraphs M-T, comprising an alkaline earth metal to aluminum atomic ratio ≥0.03, preferably ≥0.05, more preferably ≥0.06, and even more preferably ≥0.09.
V. The compositions according to Paragraphs M-U, comprising an alkaline earth metal to aluminum atomic ratio ≤1, preferably ≤0.75, more preferably ≤0.5, even more preferably ≤0.35, and most preferably ≤0.2.
W. The composition according to Paragraphs A-V, wherein the composition has a pH of about 3.5 to about 5.2, preferably from about 3.6 to about 5, more preferably from about 3.7 to about 4.8, and even more preferably from about 3.8 to about 4.8, as determined by the pH Test Method.
X. The composition according to Paragraphs A-W, wherein the composition has a maximum change in mass over 48 hours of >47%, preferably ≥55%, more preferably ≥65%, even more preferably ≥70%, and most preferably ≥75% according to the Water Vapor Sorption Test Method, described herein.
Y. The composition according to Paragraphs A-X, wherein the composition has a maximum change in mass over 48 hours of ≤120%, preferably ≤115%, more preferably ≤110%, and even more preferably ≤106%, according to the Water Vapor Sorption Test Method, described herein.
Z. The composition according to Paragraph C, wherein each particle comprises aluminum, chloride, and sodium.
AA. The composition according to Paragraph Z, wherein each particle further comprises a surface coating and at least a portion of the sodium is present in the surface coating, preferably >50% of the sodium is present in the surface coating, more preferably >75% of the sodium is present in the surface coating.
BB. The composition according to Paragraphs Z-AA, wherein at least a portion of the sodium is interdispersed with the aluminum and chloride.
CC. The composition according to Paragraph Z, wherein the particle is homogeneous.
DD. The composition according to Paragraphs A-CC, wherein the composition has a peak IV/III ratio of exceeding 0.75, preferably greater than or equal to 1, preferably greater than or equal to 1.5, preferably greater than or equal to 2, preferably greater than or equal to 2.5, preferably greater than or equal to 3, preferably greater than or equal to 3.25, preferably greater than or equal to 3.5, preferably greater than or equal to 3.75, and preferably greater than or equal to 3.85, according to the GPC Test Method.
EE. The composition according to Paragraphs A-DD, wherein the composition has a peak IV/III ratio of from about 1.5 to about 7.9, preferably from about 1.75 to about 5, more preferably from about 2 to about 4.5, more preferably from about 2.1 to about 4.25, more preferably from about 2.15 to about 4.1, even more preferably from about 2.25 to about 4, and even more preferably from about 2.4 to about 4.
FF. The composition according to Paragraphs A-EE, wherein the composition has a peak IV/III ratio of from about 2 to about 4, preferably from about 2.1 to about 3.75, more preferably from about 2.2 to about 3.5.
GG. An antiperspirant product, wherein the product comprises the composition according to Paragraphs A-FF, wherein the antiperspirant product is in the form of a spray, roll-on, cream, paste, foam, wipes, or stick.
HH. The antiperspirant product according to Paragraph GG, wherein the product further comprises a propellant and the product is a spray.
II. A non-therapeutic method of reducing perspiration from the surface of the human body, comprising a step of topical application of a product comprising the particulate active premix composition according to Paragraphs A-FF.
JJ. The method of Paragraph II, wherein the composition is applied to the underarms.
KK. A non-therapeutic use of an antiperspirant product comprising the particulate active premix composition according to Paragraphs A to FF for reduction of bodily perspiration.
LL. Non-therapeutic cosmetic use of the particulate active premix composition according to Paragraphs A to FF as an antiperspirant agent.
MM. A method of making a particulate antiperspirant active premix according to Paragraphs A-FF comprising:
   a. providing an aqueous solution of aluminum chlorohydrate with the formula Al₂(OH)_{3.778}Cl_{2.222} to Al₂(OH)_{5.048}Cl_{0.952};
   b. adding calcium chloride to provide a calcium to aluminum atomic ratio of at least 0.05 and adding glycine to provide a glycine to calcium molar ratio from about 1 to 2 to form an aqueous solution;
   d. adding sodium chloride at a concentration of more than 0.5% of the aqueous solution;
   e. after step (d), heating the aqueous solution at a temperature of more than 60° C. and less than 150° C., preferably less than 130° C., and more preferably less than 115° C. for more than 1 hour;
   f. after step (e), drying the aqueous solution to a plurality of particles to form a particulate antiperspirant active premix; and
   g. optionally grinding the particulate antiperspirant active premix to a Dv50 particle size less than 50 microns;
   h. optionally adding water to regenerate a solution comprising the aqueous premix solution.
NN. A method of making a particulate antiperspirant active premix according to Paragraphs A-FF comprising:
   a. providing an aqueous solution of aluminum chlorohydrate Al₂(OH)_{3.778}Cl_{2.222} to Al₂(OH)_{5.048}Cl_{0.952};
   b. adding calcium chloride to provide a calcium to aluminum atomic ratio of at least 0.05 and adding glycine to provide a glycine to calcium molar ratio from about 1 to 2 to form an aqueous solution;
   d. heating the aqueous solution at a temperature of more than 60° C. and less than 150° C., preferably less than 130° C., and more preferably less than 115° C. for more than 1 hour;
   e. after step (d), adding sodium chloride at a concentration of more than 0.5% of the aqueous solution;
   f. after step (e), drying, preferably spray drying, the aqueous solution to form a plurality of particles to form the particulate antiperspirant active premix; and
   g. optionally grinding the particulate antiperspirant active premix to a Dv50 particle size less than 50 microns;
   h. optionally adding water to regenerate a solution comprising the aqueous premix solution.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A particulate active premix composition comprising:
a. a basic aluminum chloride salt of formula Al₂(OH)_{3.778}Cl_{2.222} to Al₂(OH)_{5.048}Cl_{0.952}; b
b. an amino acid;
c. an alkaline earth metal salt;
d. greater than 1% of an alkali metal salt and/or magnesium chloride; and
wherein the composition comprises a plurality of particles.

2. The composition of claim 1, wherein the composition comprises up to 25% of the alkali metal salt.

3. The composition of claim 2, wherein the composition comprises an alkali metal salt chosen from sodium chloride, potassium chloride, lithium chloride, or mixtures thereof.

4. The composition of claim 3, wherein the alkali metal salt comprises sodium chloride and wherein each particle comprises aluminum, sodium, and chlorine.

5. The composition of claim 4, wherein each particle further comprises a surface coating and at least a portion of the sodium is present in the surface coating.

6. The composition of claim 1, wherein the composition comprises from about 5% to about 25% of the amino acid.

7. The composition of claim 1, wherein the amino acid is chosen from alanine, arginine, asparagine, aspartic acid, beta-alanine, trimethylglycine, cysteine, glutamic acid, glutamine, glycine, histidine, isoleucine, leucine, lysine, methionine, phenylalanine, proline, serine, threonine, tryptophan, tyrosine, valine, or mixtures thereof.

8. The composition of claim 1, wherein the alkaline earth metal salt is chosen from calcium chloride, calcium nitrate, calcium sulfate, strontium chloride, strontium sulfate, strontium nitrate, or mixtures thereof.

9. The composition of claim 1, wherein the basic aluminum chloride salt comprises aluminum chlorohydrate salt of formula Al₂(OH)_{5.05}Cl_{0.95} to Al₂(OH)_{4.95}Cl_{1.05}.

10. The composition of claim 1, wherein the basic aluminum chloride salt comprises aluminum sesquichlorohydrate salt having an aluminum to chloride atomic ratio of from 1.26 to 1.90.

11. The composition of claim 1, wherein the basic aluminum chloride salt comprises aluminum dichlorohydrate salt having an aluminum to chloride atomic ratio of from 0.90 to 1.25.

12. The composition of claim 1, wherein the composition has a maximum change in mass over 48 hours of >47% and ≤120%.

13. The composition of claim 1, wherein the composition has a peak IV/III ratio of from about 0.75 to about 7.9.

14. An antiperspirant product, wherein the product comprises the composition according to claim 1, wherein the antiperspirant product is in the form of a spray, roll-on, cream, paste, foam, wipes, or stick.

15. The antiperspirant product of claim 14, wherein the product further comprises a propellant and the product is a spray.

16. A method of making a particulate antiperspirant active premix comprising:
a. providing a basic aluminum chloride salt with the formula Al₂(OH)_{3.778}Cl_{2.222} to Al₂(OH)_{5.048}Cl_{0.952};
b. adding calcium chloride to provide a calcium to aluminum atomic ratio of at least 0.05 and adding an amino acid to provide a glycine to calcium molar ratio from about 0.5 to 5 to form an aqueous solution;
c. adding an alkali metal solution to the aqueous solution at a concentration of more than 0.5% to form the aqueous premix solution;
d. heating the aqueous premix solution at a temperature of more than 60° C. for more than 1 hour;
e. after step (d), drying the aqueous premix solution to a plurality of particles, forming the particulate antiperspirant active premix;
f. optionally grinding the particulate antiperspirant active premix to a Dv50 particle size less than 50 microns; and
g. optionally adding water to regenerate a solution comprising the aqueous premix solution.

17. A method of making a particulate antiperspirant active premix comprising:
a. providing an aqueous solution of aluminum chlorohydrate Al₂(OH)_{3.778}Cl_{2.222} to Al₂(OH)_{5.048}Cl_{0.952};
b. adding calcium chloride to provide a calcium to aluminum atomic ratio of at least 0.05 and adding glycine to provide a glycine to calcium molar ratio from about 1 to 2 to form an aqueous solution;
c. heating the aqueous solution at a temperature of more than 60° C. and less than 150° C., preferably less than 130° C., and more preferably less than 115° C. for more than 1 hour;
d. after step (c), adding sodium chloride at a concentration of more than 0.5% of the aqueous solution;
e. after step (d), drying, preferably spray drying, the aqueous solution to form a plurality of particles to form the particulate antiperspirant active premix;
f. optionally grinding the particulate antiperspirant active premix to a Dv50 particle size less than 50 microns; and
g. optionally adding water to regenerate a solution comprising the aqueous premix solution.
